(19) United States
(12) Patent Application Publication
(10) Pub. No.: US 2002/0091426 A1
(43) Pub. Date: Jul. 11, 2002

(54) Method for inducing hypothermia for treating cancer

(76) Inventor: James Allan Fox, Palo Alto, CA (US)

Correspondence Address: Roberta L. Robins, ROBINS & PASTERNAK LLP, 90 Middlefield Road, Suite 200, Menlo Park, CA, 94025, US

(21) Appl. No.: 10/058,642

(22) Filed: Jan. 28, 2002

Related U.S. Application Data
(63) Continuation of application No. 09/523,829, filed on Mar. 13, 2000.
Application No. 09/523,829 is a continuation-in-part of application No. 08/909,752, filed on Aug. 12, 1997, now Pat. No. 6,090,132.

(30) Foreign Application Priority Data
Aug. 10, 1998 (US) PCT/US98/16522
Aug. 10, 1998 (EP) 989431838

(51) Int. Cl. A61F 7/00

(52) U.S. Cl. 607/96; 607/116

(57) ABSTRACT

In the present invention heat is directed to the hypothalamus of a warm-blooded animal, utilizing the physiological mechanisms that regulate body temperature to effect a compensatory cooling response, thereby lowering body temperature. The invention relates generally to methods of treating cancer comprising inducing hypothermia by directing heat to the hypothalamus, optionally maintaining cancerous tissue at or near to normal body temperature, and optionally applying another cancer treatment. This other cancer treatment may be radiation therapy, chemotherapy, a combination of radiation and chemotherapy, or some other cancer treatment.

### CROSS-REFERENCE TO RELATED APPLICATION

This application is a continuation-in-part of U.S. patent application Ser. No. 08/909,752 filed Aug. 12, 1997, from which priority is claimed under 35 U.S.C. §120 and which application is incorporated herein by reference in its entirety.

### TECHNICAL FIELD

The invention relates generally to methods for treating cancer. More particularly, it relates to methods for treating cancer by inducing hypothermia comprising warming the hypothalamus of an animal with cancerous tissue, inducing hypothermia, and optionally maintaining the cancerous tissue at near to, or above, normal body temperature.

### BACKGROUND

Cancer is progressive disease characterized by malignant cells that grow without control, forming tumors, and often metastasize, spreading new tumors to multiple locations in the body. Treatments for cancer are often aimed at slowing cancerous growth, or reducing metastasis, or rendering cancerous tissue more susceptible to cytotoxic agents.

Hypothermia can be useful in the treatment of cancer. For example, lowered body temperature has been reported to reduce tumor metastasis in rats (Fisher et al. Archives of Surgery 98:347-351 (1969)) and in rabbits (Mandrik, Bulletin of Experimental Biology and Medicine (USSR) 47:66-70 (1959)). As long ago as 1940, Smith and Fay (American Journal of Clinical Pathology 10:1-12) reported that generalized hypothermia (as low as 74° F., with patients maintained in the low 80s F. for up to 5 to 8 days) in cancer patients led to a reduction in pain and “regressive changes in embryonic cells, particularly in carcinoma” (Id. at page 10).

Hypothermia has been combined with other cancer treatments. Harrison (Journal of Laryngology and Otology 81:173-185 (1967)) treated 11 patients with head and neck cancer with chemotherapy during whole-body hypothermia by immersion in an ice bath, and suggested this be the treatment of choice for such patients. Scaly et al. (British Journal of Radiology 59(707):1093-1098 (1986)) reported that 10 of 21 mouth cancer patients were free of disease one year after treatment with hyperbaric oxygen and radiation with hypothermia.

Another, related strategy has been to lower the temperature of an animal with a tumor or tumors while locally maintaining the temperature of the cancerous tissue near normal body temperature. Regression and disappearance of tumors in hamsters made hypothermic has been reported where the tumors were artificially maintained at normal body temperature (Popovic and Masironi, American Journal of Physiology 211:463-466 (1966); Popovic and Masironi, Cancer Research 26:863-864 (1966)). Inducing hypothermia in animals with cancer while maintaining tumor tissue normothermic has enhanced the efficacy of chemotherapeutic agents. Popovic and Masironi (Cancer Research 26(1):2353-2356 (1966)) report the regression and disappearance of tumors in hamsters treated with at a 50 mg/kg dose of 5-fluoruracil (5-FU) during hypothermia, with the tumors maintained at normal body temperature, while the same dose of 5-FU did not affect tumor size when given to tumor-bearing animals that were not made hypothermic, or hypothermic animals whose tumors were also hypothermic. Thus, the combination of hypothermia, normothermic tumors, and chemotherapy (the tumors being maintained locally at normal body temperature during application of chemotherapy) has been found to be successful in treating cancer in laboratory animals.

Methods for maintaining cancerous tissue near to or above normal body temperature in a hypothermic animal are the same as those used to locally warm tissues, and have been known for many years. For example, radiofrequency electromagnetic heating for localized tissue heating in cancer therapy was described in 1962 by Shingleton et al. (Annals of Surgery 156:408-416). More modern methods include application of ultrasound heating, as for example, may be used to heat the prostate (see, e.g., U.S. Pat. No. 5,895,356 to Andrus et al., Apparatus and method for transurethral focused ultrasound therapy). Other methods include application of warm fluids, application of warm probes, application of radiation, such as infrared, microwave, or ultrasound radiation, inductive heating, and other means.

Body temperature is very well regulated in warm-blooded animals. However, the methods and pharmacological compounds that may lower the body temperature of a laboratory rat or other small animal (with large body surface areas compared to their small volumes) do not effectively produce hypothermia in larger animals such as humans. Cooling humans and other large animals present difficulties due to our larger mass, smaller surface area in proportion to our volume, and our complex homeostatic mechanisms geared towards maintaining our body temperature.

Although difficult, it is possible to cool large animals and humans. Hypothermia (the condition of lower-than-normal body temperature in a warm-blooded animal) has been investigated in animals for many years, and has been used on human patients (for example, in heart surgery) for more than forty years. Present methods for inducing hypothermia include externally applied cold packs, ice blankets, infusion of cold saline into arteries and into the peritoneum of an animal, blowing air across an animal's skin, wetting the skin or hair of an animal, and cooling the air around an animal. Hypothermia may also be a side-effect of general anesthesia during surgery.

However, these methods often induce discomfort in the animal or are invasive. In addition, drastic measures often need be taken to overcome the normal operation of the animal's physiological responses to cold. These responses include vasoconstriction, shunting of blood away from the limbs and retention of blood in the body core (away from cold blankets, wet skin, etc.) and shivering. Suppression of these responses by muscle relaxants, vasodilators and other drugs may also cause, as side-effects, suppression of other vital body functions associated with breathing, maintenance of blood pressure, heart rate, and other vital bodily functions. These side-effects, such as circulatory shock, may be serious. They increase risk and limit the effectiveness of hypothermia treatments in humans. Thus, there is at present no simple, effective method for inducing and maintaining hypothermia in an animal.

All of the above-mentioned methods for inducing hypothermia, with the possible exception of some potent centrally-active drug regimens that disable thermoregulation, must work to oppose the animal's bodily efforts to maintain body temperature. Thus, these techniques suffer from the disadvantage that the animal's normal physiological responses are actively opposing the efforts to induce hypothermia. The homeostatic mechanisms and physiological responses involved in regulation of body temperature are among the most basic responses in warm-blooded animals. Cooling by cold blankets and dressings is uncomfortable, induces shivering which must be opposed by medication, and causes vasoconstriction which reduces blood flow to the cooled extremities, reducing the effectiveness of the cooling treatment. Cooling by intra-arterial infusion of cold blood or saline is invasive, of limited utility because of limited ability to deliver large volumes of cooled fluid, requires medical equipment and supervision, and may potentially cause vascular, cardiac and neurological (if emoboli are created) side-effects. Pharmaceutical treatments that disable thermoregulatory responses often have other effects as well, and require active cooling measures such as those already mentioned in order to lower body temperature of a large animal. Thus, an ideal method for inducing hypothermia in a warm-blooded animal would not require drastic invasive measures or drugs, and would not oppose the animal's physiological temperature control mechanisms, but would make use of them to achieve hypothermia. Accordingly, improved methods of inducing hypothermia in larger animals, including human patients, is desired.

### OVERVIEW OF RELATED ART

The following references relate to one or more aspects of the present invention:

U.S. Pat. No. 4,750,493 to Brader is directed to a method for cooling the extracranial area including the face during emergency care of cardiac arrest of severe shock in order to induce vasoconstriction and intracranial hypothermia. This invention is implemented by a topical cold pack described in the patent. This method of cooling does not directly cool the hypothalamus, nor would it trigger a physiological cooling response if it did. Instead, the physiological response of the hypothalamus to such extracranial cooling would be to oppose body cooling.

U.S. Pat. No. 4,920,963 to Brader is also directed to a method and apparatus for cooling the extracranial area including the face during emergency care of cardiac arrest of severe shock, and discloses an apparatus which includes a watertight shroud for the head. Again, this patent would not directly cool the hypothalamus, and if it did, would tend to elicit a physiological response that would act to oppose body cooling.

U.S. Pat. No. 5,383,854 to Safar, Strezoski and Klain is directed to a cardiopulmonary bypass apparatus adaptable to include a module that includes a heat exchanger capable of cooling the blood.

U.S. Pat. No. 5,464,834 to Peglion, Goument, Millan and Rivet is directed to chemical compounds acting at a 5-HT_{1A} receptor capable of inducing hypothermia in rats.

U.S. Pat. No. 5,474,533 to Ward, Brown and Dzwonczyk is directed to a method and apparatus for treating patients suffering from cardiac arrest, shock, respiratory failure, hypothermia, hyperthermia, and head injury, capable of modulating a patient's body temperature.

U.S. Pat. No. 5,486,204 to Clifton is directed to a method for treating severe brain trauma with hypothermia. Hypothermia in human patients was induced by wrapping patients in cooling blankets, and administering drugs such as muscle relaxants and sedatives.

### SUMMARY OF THE INVENTION

This invention relates to a method for treating cancer in an animal with hypothermia. The method also relates to locally warming cancerous tissue during global induction of hypothermia in an animal, and also optionally includes treatment of the animal with radiation and/or cancer chemotherapy. In each embodiment of this method, heat is directed to the hypothalamus of the animal thereby inducing hypothermia in the animal. In one embodiment, hypothermia is induced in the animal by directing heat to the hypothalamus, thereby reducing pain and reducing metastasis of cancerous tissue in the animal. In another, preferred embodiment, of the invention, hypothermia is induced in the animal by directing beat to the hypothalamus, while at the same time cancerous tissue is maintained at or near to normal body temperature. In a more preferred embodiment of the invention, hypothermia is induced in the animal by directing heat to the hypothalamus, while at the same time cancerous tissue is maintained at or near to normal body temperature, and another cancer treatment is applied. This other cancer treatment may be radiation therapy, chemotherapy, a combination of radiation and chemotherapy, or some other cancer treatment.

Known methods for inducing hypothermia all involve cooling the outside or inside of an animal, sometimes in conjunction with drugs that disable the animal's homeostatic responses. It is new and unsuggested in the art to direct heat to the hypothalamus in an effort to reduce body temperature. The present invention is directed to a method and apparatus for directing heat to the hypothalamus of a warm-blooded animal in order to utilize the physiological mechanisms that regulate body temperature to effect a compensatory cooling response, thereby lowering body temperature. This method may be used alone, or in conjunction with other methods for inducing hypothermia; when used with other methods, it will serve to improve their efficacy, by taking advantage of physiological temperature-regulatory mechanisms and making direct use of their action, instead of striving to oppose or disable them.

In one aspect of the invention, hypothermia itself is used to treat cancer in a patient. In another aspect, systemic hypothermia combined with application of heat to the cancerous tissue to maintain the temperature of the cancerous tissue near to, or above, normal body temperature, is used to treat cancer in a patient. In a third aspect of the invention, while systemic hypothermia is induced by directing heat to the hypothalamus, local heating is applied to cancerous tissue, maintaining that tissue near to, or above, normal body temperature, and radiation or chemotherapeutic agents are administered to the patient. Means for maintaining the temperature of cancerous tissue during systemic hypothermia include heating supplied by warm probes, warm liquids, hot gases, electromagnetic radiation, ultrasound, direction of warm blood flow to an isolated limb, or other means applied locally or directed to the cancerous tissue.

It is well-known that the main brain center for regulation of body temperature is in the hypothalamus, a brain structure situated in humans just above the pituitary gland. Decreasing the temperature of the hypothalamus, as occurs when core body temperature is reduced, triggers compensatory responses to cold, such as vasoconstriction and shivering. Conversely, warming the hypothalamus triggers compensatory responses that cool the animal, such as vasodilation and sweating. Thermoregulatory responses can be quite effective, as humans routinely live and work in environments where the external temperature is higher or much lower than normal body temperature.

The hypothalamus is very sensitive to small changes in body temperature. A temperature change of 0.2 degrees Celsius (° C.) is sufficient to trigger sweating in a human subject. Sweating is a major mechanism for cooling in humans. Sweating will continue as long as the hypothalamic temperature is above its setpoint for temperature control. Thus, for example, if the temperature of the hypothalamus in a human patient is raised to about 0.2° C. or more above its setpoint, the patient will respond with such physiological cooling responses as vasodilation and sweating. These responses may continue indefinitely in response to sustained raised temperature of the hypothalamus. These physiological cooling responses will act to lower the body temperature of the patient.

In humans, the hypothalamus is located near to the sphenoid sinus, one of the sinuses accessible through the nose or mouth. Heat may be applied via the sinuses, or more particularly to the sphenoid sinus, in order to warm the hypothalamus and so to trigger a cooling response. Alternatively, heat may be applied directly to the hypothalamus. Heating of the hypothalamus may be accomplished with little heating of surrounding brain regions. Heat applied near the surface of the skull will penetrate a small distance, but blood flow and other thermal effects will cause the heating to be localized to the portion of the brain nearest the application of the heat. Thus, mild local heating of the sphenoid sinus or other nasal passages or sinuses in order to warm the hypothalamus would not cause undue heating of other portions of the brain. Alternatively, warming the nasal passages themselves can trigger sweating and so be effective for lowering body temperature.

Accordingly, a primary object of the present invention is to provide a method for treating a cancerous condition in a patient comprising inducing hypothermia by directing heat to the hypothalamus.

An additional object of the invention is to provide a method for treating a cancerous condition in a patient comprising inducing hypothermia by directing heat to the hypothalamus, and maintaining the temperature of the cancerous tissue near or above normal body temperature.

An additional object of the invention is to provide a method for treating a cancerous condition in a patient comprising inducing hypothermia by directing heat to the hypothalamus, and maintaining the temperature of the cancerous tissue near or above normal body temperature, and administering radiation treatment or a chemotherapeutic agent.

Additional objects, advantages and novel features of the invention will be set forth in part in the description which follows, and in part will become apparent to those skilled in the art upon examination of the following, or may be learned by practice of the invention.

In one aspect, the invention is a method for inducing hypothermia which comprises providing a heating means, directing heat to the hypothalamus of an animal, resulting in a physiological response effective to lower the body temperature of the animal so treated.

In another aspect, this invention is an apparatus for directing heat to the hypothalamus of an animal, the use of which apparatus results in a physiological response effective to lower the body temperature of the animal so treated.

In still another aspect, the invention is a method for inducing hypothermia which comprises providing a heating means and, with this heating means, applying heat to a nasal passage, or a nasal passage and a sinus, or a nasal passage, sinus and hypothalamus, or to a sinus and hypothalamus, or to the hypothalamus. This application of heat to a nasal passage, or a nasal passage and sinus, or sinus and hypothalamus, or hypothalamus will result in a physiological response effective to lower the body temperature of the animal so treated.

In yet another aspect, this invention is an apparatus for applying heat to a nasal passage, or a nasal passage and a sinus, or a nasal passage, sinus and hypothalamus, or to a sinus and hypothalamus, or to the hypothalamus. Application of heat through the use of said apparatus to a nasal passage, or a nasal passage and a sinus, or a sinus and the hypothalamus, or the hypothalamus will result in a physiological response effective to lower the body temperature of the animal so treated.

In a preferred embodiment, thermal energy is directed towards the hypothalamus, warming the hypothalamus and producing a therapeutic cooling response. Such thermal energy may be directed by direction of electromagnetic radiation towards the hypothalamus, or placement of a warm probe near to the hypothalamus, or placement of a warm probe in or near to the hypothalamus, or placement, near to or in the hypothalamus, of a probe capable of being heated by electrical means, by application of radiofrequency energy, by conduction of heat, or other heating means.

The cooling response induced by such hypothalamic warming is useful for reducing body temperature, alone or in conjunction with other methods for inducing hypothermia, and is used in the present invention as a treatment for cancer or as an adjunct to cancer therapy.

### DETAILED DESCRIPTION OF THE INVENTION

Definitions.

Before the present methods and apparatus are disclosed and described, it is to be understood that this invention is not limited to specific methods, means and apparatus, as methods for applying heat, means for directing the heat so applied, and apparatus for applying heat to a nasal passage, sinus, brain region or anatomical structure may, of course, vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly states otherwise. Thus, for example, reference to “a sinus” includes more than one sinus.

The term “animal” is meant to include humans and other warm-blooded animals.

By the term “hypothermia” is meant the condition of lower-than-normal body temperature in a warm-blooded animal, that is, a reduction in, or lowering of, body temperature in an animal. Preferably, body temperature in hypothermia is lowered by at least about 2° C. to about 3° C. (that is, to between about 34° C. to about 35° C.), or more preferably, by at least about 3° C. to about 5° C. (that is, to between about 32° C. to about 34 ° C.), most preferably by at least about 5° C. (that is, to below about 32° C.).

The term “heat” is used in its conventional sense to mean thermal energy content or warmth; the verb-form of the term, “to heat,” is used herein to mean to transfer thermal energy, to increase the temperature of, to warm.

The term “hypothalamus” as used herein is meant to include the anatomical region of the brain termed in standard neuroanatomical usage the hypothalamus, but is not meant to be strictly limited to this neuroanatomical region. As used herein, the term “hypothalamus” is meant to include the brain regions generally accepted as being important in the regulation of body temperature in warm-blooded animals, particularly the pre-optic and suprachiasmatic nuclei of the hypothalamus, including neighboring areas of the brain such as the septum that have been found to be important in thermoregulation.

The terms “nasal passage” and “nasal passages” are used herein to include the nostrils and to mean the anatomical regions connecting the nostrils with the sinuses of the skull.

The term “oral passage” is meant herein to include the mouth and throat, and the opening connecting the oral cavity with the nasal passages behind the palate.

By the term “physiological cooling response” is meant the physiological and behavioral responses of an animal to warming, or to stimuli that usually accompany warming, usually effective to cool the animal. Responses that are usually effective to cool an animal are effective to rid the animal of excess heat or to reduce the body temperature of the animal, and may include, but are not limited to sweating, peripheral vasodilatation, panting, drooling, licking, and repositioning the body.

The term “cancerous tissue” refers to cells, or groups or aggregates of cells, that are cancerous.

The term “cancerous condition” refers to the condition of an animal with cancerous tissue, such as cancerous cells or a tumor, or other cancer.

The term “normal body temperature” refers to temperature of about 37° C., that is, preferably between about 35° C. to about 40° C. most preferably between about 36° C. to about 38° C.

### BRIEF DESCRIPTION OF THE DRAWINGS

FIG. 1 is a cross-sectional view of a human head, showing placement of a tube in a nostril.

FIG. 2 is a cross-sectional view of a human head, showing placement of a warming device into the sphenoid sinus.

FIG. 3 is a cross-sectional view of a dual-lumen catheter with an occluded end.

FIG. 4 is a cross-sectional view of an electrically-heated device with a thermal sensor.

FIG. 5 is a cross-sectional view of a human head, showing placement of heating means into the hypothalamic region of the brain.

FIG. 6 is a cross-sectional view of a three-lumen catheter for mixing fluids to produce heat near the tip.

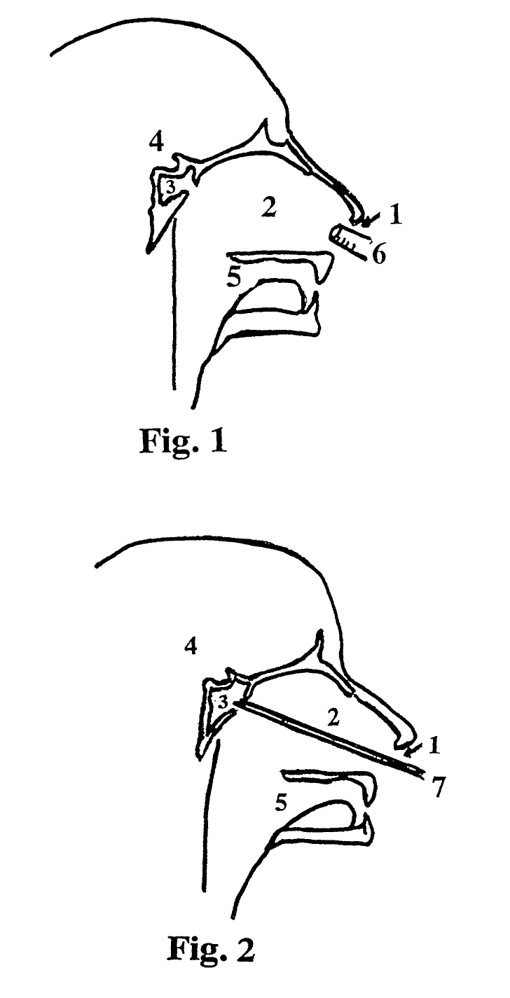

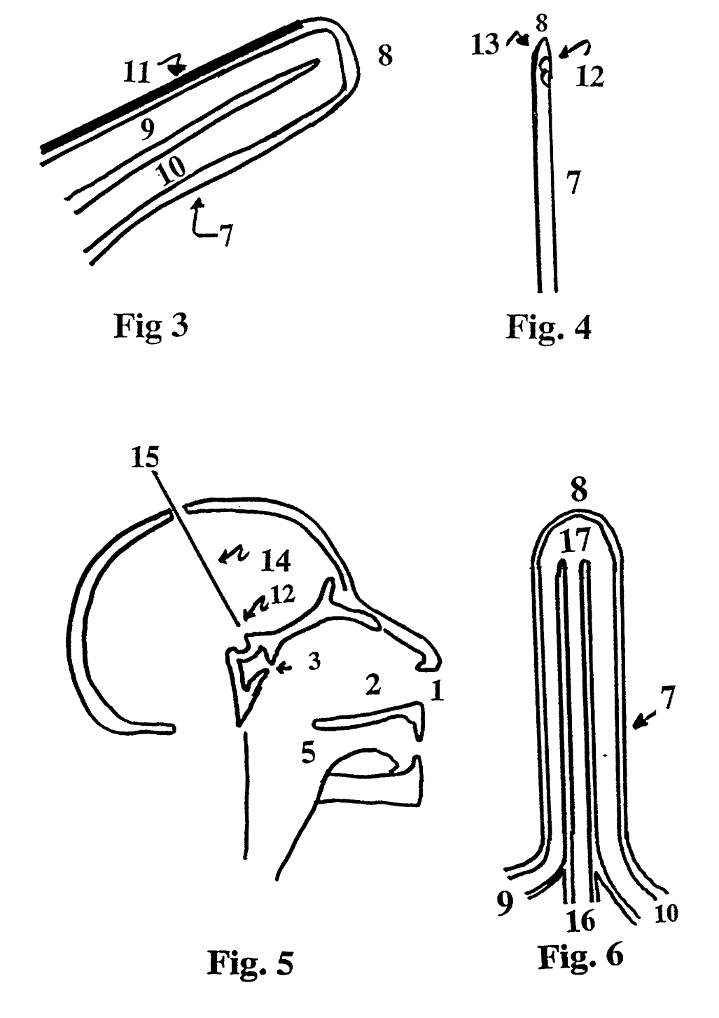

### DETAILED DESCRIPTION OF A PREFERRED EMBODIMENT OF THE INVENTION

Method of Use

Known methods and devices for inducing hypothermia in an animal rely on cooling methods, cooling devices or drug therapies that impair normal body temperature regulation. It is novel to propose warming to induce hypothermia. It is also known that body temperature is well-controlled in warm-blooded animals. The brain regions most important in the regulation of body temperature are in and near the hypothalamus.

It is known that small changes in hypothalamic temperature will cause physiological responses that act to restore body temperature to normal. It is novel to suggest, as is suggested in the present invention, that inducing small changes in hypothalamic temperature can be used for therapeutic effect.

Warming the hypothalamus 4 by direct application of heat, via a probe 14 placed in or near the hypothalamus, as shown in FIG. 5, or by direction of energy to the hypothalamus 4 will cause the hypothalamus to respond as if body temperature had risen, that is, by acting to lower body temperature in an attempt to restore body to normal. This hypothalamic response will continue as long as the temperature of the hypothalamus is maintained above its set point. Thus, where hypothermia is indicated for a desired therapeutic effect, warming the hypothalamus can be useful by inducing hypothermia, or by enhancing the effects of other methods for inducing hypothermia applied in conjunction with hypothalamic warming.

It is known that the hypothalamus 4 in humans is located near to the sphenoid sinus 3, a sinus which is accessible from the outside of a person via nasal passages 2 and the nostrils 1 and via the oral passage 5. A novel aspect of the present invention is the recognition that application of heat to the sphenoid sinus will warm the hypothalamus and cause a physiological cooling response. Warming of the sphenoid sinus will warm the hypothalamus of a person with normal blood flow but will not greatly warm other brain regions. Modeling suggests that the temperature near to a heat source in a well-perfused brain rises significantly only at the points nearest the heat source. Thus, application of heat to a nasal passage, sinus or directly to the hypothalamus will have only local direct effects on brain temperature, will not significantly raise temperature in other, more distal, brain regions, and will lead to global hypothermia.

The exact parameters of warming a nasal passage, sinus or hypothalamus, or combinations of these, may vary, as will be appreciated by those skilled in the art of medical practice, but will necessarily involve providing a warming means, applying said warming means so as to warm the hypothalamus or sinus or nasal passages, or combinations of these, to between about 38° C. and about 50° C. As said warming means is being applied, compounds may be introduced into a nasal passage or sinus. In some cases, it may be desirable as well to apply cooling measures to the animal, such as blowing air across exposed skin, applying cold dressings to exposed skin, and so forth. However, moderate cooling measures, if any, are preferred, since lowering the skin temperature will raise the hypothalamic temperature set-point. For this reason, it may be advantageous to warm portions of the skin in order to lower the hypothalamic set-point and so aid in maintaining a lowered body temperature.

Description of Apparatus of the Invention

Heat may be applied to a nasal passage or a sinus or to a nasal passage and a sinus through the breathing of a warm gas, such as air mixed with steam or water mist (in a ratio of approximately 0% to 40% by volume) at a temperature between about 38° C. and about 50° C., although other temperatures may also be effective. Preferred temperatures are between about 38° C. and 43° C. This gas may be supplied, for example, to a nasal passage via a hollow tube 6, of a size smaller than a human nostril. Tubes effective for this purpose are approximately 0.1″ to approximately 0.5″ in outer diameter, may be thin-walled or thick-walled, and may be made, for example, of Tygon tubing. This tube may be inserted a short distance (for example, less than 0.5″) into a nasal passage, or may be inserted farther into a nasal passage (for example, approximately 1″ or more). Care must be taken that the animal breathes sufficient oxygen for health, and that sensitive nasal tissue is not scalded. If higher temperature gases are used, or higher fractions of steam or other warm gas, then smaller diameter tubes which do not fully occlude the nostril and so allow passage of air into the nasal passage are preferred. If lower temperature gases, nearer to 38° C. than 50° C., are used, then larger diameter tubing which occludes the nostril may be used.

Similarly, heat may be applied to a sinus, preferably the sphenoid sinus, through direction of a heated gas such as air mixed with steam via a tube or catheter 7. This heated gas may be air mixed with steam (in a ratio of approximately 0% to 40% by volume) at a temperature between about 38° C. and about 50° C., although other temperatures may also be effective. Preferred temperatures are between about 38° C. and 43° C. This gas may be supplied, for example, to a sinus by a hollow tube with an outside diameter of between about 0.05″ and about 0.25″. This hollow tube may be thin-walled or thick-walled, and is made, for example, of about 10 cm of flexible tubing with a smooth 4 mm curve at the distal end. This tube may be inserted through a nostril or through the mouth and oral cavity to gain access to a nasal passage above the palate and then into a sinus. Insertion of this tube may be aided by a guide tube, a guide wire, or other implement. Care must be taken that sensitive tissue is not scalded or damaged during insertion. Insertion of this tube is preferably done using methods in common use for introduction of drain tubes into inflamed sinuses for the purpose of draining accumulated fluids.

A nasal passage or sinus may be also warmed by an apparatus which is itself heated and delivers heat. One such apparatus comprises a closed-ended flexible tube containing warm gases, such as the mixtures of steam and air, or other warm gases, or preferably containing a warm fluid, such as warm saline or other liquid, capable of being introduced into a nasal passage or sinus. Warm fluids are preferred over warm gases in this embodiment because their higher heat capacity and greater mass make them more effective to warm the tissue with which they are in contact. The temperature of said gas or fluid may be between about 38° F. and 50° C. Preferred temperatures are between about 38° C. and 43° C. Such an apparatus can be a tube with a single lumen and a distal end blocked to prevent outflow of hot gases or fluids. In a preferred embodiment, this apparatus comprises a tube with at least two inner lumens, at least one for inflow of warm fluid or gas 9, at least one for outflow of warm fluid or gas 10, with a distal end of said tube blocked to prevent outflow of the warm fluid or gas enclosed 8. Constant flow of said warm fluid or gas is maintained to provide continuous heating to the nasal passage or sinus. In a preferred embodiment, said closed end is a distal end comprised of thinner wall thickness than the lateral wall of the apparatus. An effective thickness for the blocked distal end is between about 0.001″ and about 0.05″. In another preferred embodiment, said distal end is constructed of a thin flexible material, such as latex rubber or polyethylene of a thickness between about 0.001″ and about 0.05″ effective for transferring heat and capable of expanding or “ballooning out” to fill space surrounding it under application of internal positive pressure. This apparatus may be inserted through a nostril or through the mouth and oral cavity and then to a nasal passage above the palate and into a sinus. Insertion of this apparatus may be aided by a guide tube, a guide wire 11, or other implement. Care must be taken that sensitive tissue is not scalded or damaged during insertion. Insertion of this apparatus is preferably done using methods in common use for introduction of drain tubes into inflamed sinuses for the purpose of draining accumulated fluids.

Another such apparatus effective to warm a nasal passage or sinus comprises an electrical warming device 12 attached at an end of a flexible tube, rod or catheter capable of being introduced into a nasal passage or sinus. Said electrical warming device may be a thermocouple, Peltier device, electrical heating element, or the like. In a preferred embodiment the electrical warming is obtained by passing electrical current through an insulated coil of nichrome wire (30 to 36 AWG, coil outer diameter 0.04″) connected to insulated copper or silver wires (26 to 30 gauge, twisted wire) insulated with a flexible insulating coating of an insulating material such as polyimide or epoxy. The heating element is preferably shaped in a helical coil with an outer dimension of about 0.08″ diameter and contained inside an insulating coating. Effective temperatures at the heating element are between about 38° C. and about 50° C. Preferred temperatures are between about 38° C. and about 43° C. to provide warming of the nasal passage or sinus. In a preferred embodiment, a temperature sensor 13 is enclosed with the coil to provide temperature feedback to control the applied temperature. This apparatus may be inserted through a nostril or through the mouth and oral cavity and to a nasal passage above the palate and then into a sinus. Insertion of this apparatus may be aided by a guide tube, a guide wire, or other implement. Care must be taken that sensitive tissue is not scalded or damaged during insertion. Insertion of this apparatus is preferably done using methods in common use for introduction of drain tubes into inflamed sinuses for the purpose of draining accumulated fluids.

Heating of the hypothalamus directly may be accomplished by insertion of heated tubes or electrical devices, and other devices of similar effects, directly into the hypothalamus though surgical procedures. Preferred apparatus are not flexible tubes, but are made of medical grade stainless steel 14, of an outside diameter between about 0.01″ and about 0.08″. Alternatively, flexible wires may connect to heating elements placed in or near the hypothalamus. FIG. 5 illustrates one surgical approach for placement of a heating probe into the hypothalamus. Other approaches are suitable as well, including, for example, introducing probes or heating elements near or in the hypothalamus from surgical entry points at the side of the skull, or via a frontal approach. Electrical methods of heating are preferred over methods utilizing heated fluids or gases for this embodiment of the invention. Preferred electrical methods and apparatus include heating elements such as thermocouples, Peltier devices and resistive heating wires such as Nichrome wire provided at the tips of stainless steel rods or attached to flexible wires. In addition, the delivery of radio-frequency current is effective to warm the hypothalamus. Electrical stimulation of neurons in the hypothalamus is also effective to stimulate hypothalamic neurons to trigger a physiological cooling response.

Heating of the hypothalamus may be effected by infrared radiation directed to the hypothalamus, such as infrared radiation delivered to the inside of the sphenoid sinus or nasal passage by an infrared source such as a heated coil inside a thermally cooled jacket. In this embodiment, warming of the hypothalamus is effected by either infrared radiation alone, or by infrared radiation along with heat delivered to the hypothalamus by conduction through intervening tissue. Other forms of radiation and radiative heating are suitable as well.

Physiological cooling responses may be initiated by introduction of chemical compounds into a nasal passage and a sinus, at the same time as warm gases or heat is introduced, or in the absence of said heating. Compounds such as melatonin, capsaicin and other compounds are effective to induce a physiological cooling response. Effective concentrations of melatonin are between about 0.1 nM and about 100 nM. Effective concentrations of capsaicin are between about 1 nM and about 1 μM.

Local warming of cancerous tissue, during hypothermia, may be accomplished by application of a heated probe, such as probe 14 (described above for use in the hypothalamus) shown in FIG. 5, or application of heated fluids, hot gas (such as warm or hot air, or other gases), heating pads, electric heaters, tubes or containers containing heated fluids, or the like. Alternatively, thermal radiation, whether infrared, microwave, or other radiation, may be directed to the cancerous tissue to maintain its temperature near to, or above, normal body temperature while the rest of the body is hypothermic. Similarly, ultrasound radiation may be applied to maintain the temperature of the cancerous tissue near to, or above, normal body temperature while the rest of the body is hypothermic. That is, during hypothermia, when substantially all of the rest of the animal experiences reduced body temperature, the temperature of the cancerous tissue is maintained at a temperature of between about 35° C. and about 44° C., preferably between about 35° C. to about 40° C., and most preferably between about 36° C. to about 38° C.

When the treatment includes chemotherapy, standard chemotherapy regimens known in the art are applied. Chemotherapy regimens may use standard or nonstandard chemotherapy drugs including as methotrexate, 5-fluoruracil, doxorubicin, cisplatin, taxol, and other drugs, individually or in combination. The dosage of drug or drugs used can depend upon various factors, such as the mode of administration, species, age, weight and individual state. The doses to be administered daily are about 0.05 to about 100 mg/kg body weight per individual component. The amount of the particular active material per form of administration can be from about 5 to about 1000 mg.

When the treatment includes radiation treatment, standard radiation therapy regimens known in the art are applied. Typical radiotherapy regimens include exposure of cancerous tissue to ionizing radiation daily (e.g., five times per week) for about 5 or more weeks, where both the length of treatment and the total dosage of radiation are dictated by the tumor size, location, tumor type, and other factors known in the art. For example, treatment regimens for small microscopic tumors typically include daily exposure of the tumor to ionizing radiation for approximately 5 weeks with a cumulative ionizing radiation dosage of about 4500-5000 rads. Treatment regimens for larger tumors as well as tumors located in the head and neck typically are extended to 8 or more weeks and can employ a cumulative ionizing radiation dosage of about 7400 or more rads.

It is to be understood that while the invention has been described in conjunction with preferred specific embodiments thereof, the foregoing description, as well as the examples which follow, are intended to illustrate and not limit the scope of the invention. Other aspects, advantages and modifications within the scope of the invention will be apparent to those skilled in the art to which the invention pertains. All patents, patent applications and publications mentioned herein, both supra and infra, are incorporated by reference in their entirety.

### EXAMPLE 1

This example illustrates the use of warm gas to warm a nasal passage, sinus and hypothalamus. A mixture of 25% steam and 75% air (v/v) is combined in a chamber to which are connected two flexible Tygon tubes of outer diameter 0.125″ which are inserted into the nostrils of a human subject. The human subject breathes the warm gas mixture normally through the nose. In approximately 5 minutes, the subject begins to sweat. This physiological cooling response is effective to lower the animal's body temperature.

### EXAMPLE 2

This example illustrates the use of a warming tube inserted into the sphenoid sinus via a nostril. The warming tube is a flexible tube with two lumens. Warm saline (43° C.) at a pressure head of 50 cm flows towards the distal end of the warming tube in one lumen, and returns via the other lumen at a pressure head of 0 cm. The distal end of the warming tube opens into and is enclosed by a distensible balloon made of latex rubber (wall thickness 0.005″) which acts to direct the warm fluid flow from the inflow tube to the outflow tube. A stainless steel guide wire 11 cm in length with a shapeable tip, gently curved for the last 4 mm, is inserted into the guidewire lumen and is used to direct the distal end of the warming tube into the sphenoid sinus. The latex rubber balloon at the distal end of the warming tube expands into the sphenoid sinus once the outflow pressure head becomes positive following partial occlusion of the outflow. The circulation of warm saline inside the balloon warms the sphenoid sinus, the skull and the brain structures adjacent the sphenoid sinus, including the hypothalamus, and the animal begins to sweat. This physiological cooling response is effective to lower the animal's body temperature.

### EXAMPLE 3

This example illustrates the use of warm gas to warm a nasal passage, sinus and hypothalamus and the additional use of compounds effective to increase the hypothermia induced by warming a nasal passage, sinus and hypothalamus. A mixture of 25% steam and 75% air (v/v) with added melatonin and capsaicin is combined in a chamber to which is connected a two-lumen flexible polyethylene extrusion of outer diameter 4 mm which is inserted into the nostrils of a human subject. The melatonin and capsaisin are added to the mixing chamber via an atomizer spray as a solution in saline (0.9% sodium chloride) of 10 nM melatonin, 1 mg/mL bovine serum albumin and 100 nM capsaicin. The human subject breathes the warm gas mixture normally through the nose. In approximately 5 minutes, the animal begins to sweat. This physiological cooling response is effective to lower the animal's body temperature.

### EXAMPLE 4

This example illustrates the use of direct heating in the hypothalamus to produce a physiological cooling response. Standard imaging techniques are used to image the brain of the human patient prior to any surgical procedures. Accepted neurosurgical procedures are followed when exposing the skull and drilling a small hole in the skull 15, inserting a guide-tube under stereotactic control. After local anesthetic is applied, a Leksell stereotactic frame is applied to the head of a human patient and an incision in the scalp is made, exposing the skull. A single 3-mm twist drill hole is made in the exposed skull anterior to the coronal suture approximately 2 cm from the midline. The dura is penetrated with a sharp probe and a 1.1 mm guide tube is stereotactically placed in the cerebrum so that the tip is within 1 cm of the anterior hypothalamus. A sterile microelectrode is attached to a hydraulic microdrive and the tip of the electrode is advanced down the guide tube until it protrudes from the guide tube into the pre-optic/anterior hypothalamic region of the hypothalamus. Monopolar radio-frequency stimulation is imposed. The ground is 3.5 inch, 18-gauge needle placed into a deltoid muscle. Measurement of the temperature of the electrode tip is used to control the power such that the temperature rise at the electrode tip is limited to 1° C. The patient begins sweating and vasodilation begins within 5 minutes, and the patient's body temperature begins to drop.

### EXAMPLE 5

This example illustrates the use of a chemical warming device to produce a physiological cooling response in a human patient. This chemical warming device comprises a guide wire, fluid reservoirs and pumps, and a flexible polyethylene tube of outer diameter 0.1″ with three lumens: two of 0.03″ inner diameter, one of 0.015″ inner diameter 16. The distal end of this tube is occluded with all three lumens opening into a common mixing chamber 17 of 0.08″ diameter and 0.04″ breadth. Distilled water flows in both of the larger diameter lumens, one lumen serving for inflow of distilled water, the other for outflow. The third, smallest lumen contains an aqueous mixture of 100 mM KOH. Liquid in all three tubes is maintained at a pressure less than ambient pressure to insure that, in the event of failure, no liquid will escape into the patient. Liquid flow is maintained by a siphon effect, with the two inflow reservoirs and one outflow reservoir being situated below the level of the patient's head. KOH solution is introduced into the mixing chamber by increasing the flow rate of that liquid (with a concomitant increase in outflow rate). This causes the KOH solution to mix with and to be diluted in the distilled water, thereby releasing heat in the region where mixing occurs. The chemical warming device is used in the following manner: the distal end of the chemical warming device is directed near to the sphenoid sinus as distilled water is flowing through the tube; when the tube is positioned as desired, KOH solution flow is initiated, with an increase in the fluid outflow rate as well to insure adequate and appropriate removal of fluid from the mixing chamber situated in the distal end of the tube, thereby heating the tip region of the chemical warming device, warming a sinus, and inducing a physiological cooling response.

### EXAMPLE 6

This example illustrates the induction of hypothermia by directing heat to the hypothalamus as therapy for cancer. Hypothermia may be induced by any method, such as the methods of Examples 1 through 5, in a patient suffering from cancer that is susceptible to metastasis. In this example, hypothermia is induced by the method of Example 4. Heating is chronically maintained in the hypothalamus for as long as the probe is in place within the skull of the patient. Induction of hypothermia is effective to reduce the extent of metastasis of the cancer, thereby slowing the progression of the disease in the patient.

### EXAMPLE 7

This example illustrates the induction of hypothermia by directing heat to the hypothalamus as an adjunct to cancer radiation therapy. Hypothermia may be induced by any method, such as the methods of Examples 1 through 5, in a patient suffering from cancer that is susceptible to radiation treatment. In this example, hypothermia is induced by the method of Example 4. Heating of the hypothalamus, effective to induce hypothermia, is maintained for a period of five hours, beginning prior to the initiation of each radiation therapy session. Induction of hypothermia is effective to enhance the effectiveness of the radiation therapy, thereby aiding the treatment of the disease in the patient.

### EXAMPLE 8

This example illustrates the induction of hypothermia by directing heat to the hypothalamus as an adjunct to cancer radiation therapy. Hypothermia may be induced by any method, such as any of the methods of Examples 1 through 5, in a patient suffering from skin cancer that is susceptible to radiation treatment. In this example, hypothermia is induced by the method of Example 4. Heating of the hypothalamus, effective to induce hypothermia, is maintained for a period of five hours, beginning prior to the initiation of each radiation therapy session. The cancerous tissue in the patient is maintained at normal body temperature by the application to the cancerous skin of a warm pad slightly larger in area than the area of cancerous tissue. The pad is heated by circulating warm water within the pad. Induction of hypothermia while maintaining the cancerous tissue at a temperature between 36° C. and 38° C., is effective to enhance the effectiveness of the radiation therapy, thereby aiding the treatment of the disease in the patient.

### EXAMPLE 9

This example illustrates the induction of hypothermia by directing heat to the hypothalamus as an adjunct to cancer radiation therapy. Hypothermia may be induced by any method, such as any of the methods of Examples 1 through 5, in a patient suffering from prostate cancer that is susceptible to radiation treatment. In this example, hypothermia is induced by the method of Example 4. Heating of the hypothalamus, effective to induce hypothermia, is maintained for a period of five hours, beginning prior to the initiation of each radiation therapy session. The cancerous prostate tissue in the patient is maintained at normal body temperature by the direction of ultrasound energy to the cancerous prostate. The prostate is warmed by an ultrasound probe inserted via the urethra, by the method of Andrus et al., U.S. Pat. No. 5,895,356. Induction of hypothermia while maintaining the cancerous prostate at a temperature between 36° C. and 38° C. is effective to enhance the effectiveness of the radiation therapy, thereby aiding the treatment of the disease in the patient.

### EXAMPLE 10

This example illustrates the induction of hypothermia by directing heat to the hypothalamus as an adjunct to cancer chemotherapy. Hypothermia may be induced by any method, such as any of the methods of Examples 1 through 5, in a patient suffering from prostate cancer that is susceptible to chemotherapy treatment. The method of Example 4 is used to warm the hypothalamus of a patient undergoing chemotherapy for prostate cancer, effective to induce hypothermia in the patient during the time the patient receives his chemotherapy treatment. Five hours after the chemotherapy treatment session is completed, the hypothalamic warming is curtailed, and the patient's body temperature returns to normal. The chemotherapy is much more effective than such treatment in the absence of the hypothermia.

### EXAMPLE 11

This example illustrates the induction of hypothermia by directing heat to the hypothalamus as an adjunct to cancer chemotherapy. Hypothermia may be induced by any method, such as any of the methods of Examples 1 through 5, in a patient suffering from prostate cancer that is susceptible to chemotherapy treatment. The method of Example 4 is used to warm the hypothalamus of a patient undergoing radiation therapy for prostate cancer. The prostate is warmed by an ultrasound probe inserted via the urethra, by the method of Andrus et al., U.S. Pat. No. 5,895,356. The prostate itself is maintained at a temperature between 36° C. and 38° C. while the temperature of the rest of the patient is reduced to below 33° C. Five hours after the chemotherapy, the hypothalamic warming is curtailed, and the patient's body temperature returns to normal. The chemotherapy is much more effective than such treatment in the absence of the temperature differential between the cancerous tissue and the rest of the body.

I claim:

1. A method for treating cancer in an animal with a hypothalamus, comprising the steps of providing a warming means, and applying said warming means so as to direct heat to said hypothalamus, effective to cause said animal to respond to said heat with a physiological cooling response effective to induce hypothermia in said animal.

2. A method for treating cancer in an animal with a hypothalamus, said animal having cancerous tissue and a normal body temperature, comprising the steps of providing a first and a second warming means, applying said first warming means so as to direct heat to said hypothalamus, effective to cause said animal to respond to said heat with a physiological cooling response effective to induce hypothermia in said animal, and applying said second warming means effective to maintain said cancerous tissue near to said normal body temperature.

3. A method for treating cancer in an animal with a hypothalamus, said animal having cancerous tissue and a normal body temperature, comprising the steps of providing a first and a second warming means, applying said first warming means so as to direct heat to said hypothalamus, effective to cause said animal to respond to said heat with a physiological cooling response effective to induce hypothermia in said animal, applying said second warming means effective to maintain said cancerous tissue near to said normal body temperature, and applying a second cancer treatment to said animal.

4. The method of claim 3, wherein the second cancer treatment is selected from the group consisting of chemotherapy, radiation therapy, and chemotherapy combined with radiation therapy.

5. The method of claim 2, wherein the cancerous tissue is maintained at a temperature between about 35° C. and about 44° C.

6. The method of claim 2, wherein the cancerous tissue is maintained at a temperature between about 35° C. and about 40° C.

7. The method of claim 2, wherein the cancerous tissue is maintained at a temperature between about 36° C. and about 38° C.

8. The method of claim 3, wherein the cancerous tissue is maintained at a temperature between about 35° C. and about 44° C.

9. The method of claim 3, wherein the cancerous tissue is maintained at a temperature between about 35° C. and about 40° C.

10. The method of claim 3, wherein the cancerous tissue is maintained at a temperature between about 36° C. and about 38° C.

11. The method of claim 3, wherein the cancerous tissue is maintained at a temperature between about 35° C. and about 44° C. and wherein the second cancer treatment is selected from the group consisting of chemotherapy, radiation therapy, and chemotherapy combined with radiation therapy.

12. The method of claim 3, wherein the cancerous tissue is maintained at a temperature between about 35° C. and about 40° C., and wherein the second cancer treatment is selected from the group consisting of chemotherapy, radiation therapy, and chemotherapy combined with radiation therapy.

13. The method of claim 3, wherein the cancerous tissue is maintained at a temperature between about 36° C. and about 38° C. and wherein the second cancer treatment is selected from the group consisting of chemotherapy, radiation therapy, and chemotherapy combined with radiation therapy.
